(19) United States
(12) Patent Application Publication
(10) Pub. No.: US 2009/0275647 A1
(43) Pub. Date: Nov. 5, 2009

(54) TAXANE DERIVATIVE COMPOSITION

(75) Inventors: Ashish SEHGAL, Ahmedabad (IN); Bhavesh PATEL, Ahmedabad (IN); Jayanta Kumar MANDAL, Ahmedabad (IN)

Correspondence Address: HAMRE, SCHUMANN, MUELLER & LARSON, P.C., P.O. BOX 2902, MINNEAPOLIS, MN, 55402-0902, US

(73) Assignee: INTAS Pharmaceuticals Limited, Ahmedabad (IN)

(21) Appl. No.: 12/259,634

(22) Filed: Oct. 28, 2008

Related U.S. Application Data
(63) Continuation of application No. PCT/IN08/00628, filed on Sep. 29, 2008.

(30) Foreign Application Priority Data
Oct. 1, 2007 (IN) 1950/MUM/2007

(51) Int. Cl. A61K 31/337

(52) U.S. Cl. 514/449

(57) ABSTRACT

A stable, absolutely ethanol free composition of docetaxel that prevents alcoholic intoxication or anaphylactic shock. The composition can be in the form of a stable injectable composition that includes a taxane derivative, wherein the composition includes a mixture of docetaxel, one or more stabilizer, one or more surfactant, or more co-solvent and water for injection wherein the composition is absolutely free of ethanol.

### FIELD OF THE INVENTION

The present invention provides a stable, absolutely ethanol free, composition of docetaxel to prevent the patient from alcoholic intoxication or anaphylactic shock.

### BACKGROUND AND PRIOR ART

Docetaxel, characterized as low water soluble molecule, surfactant and alcohol preferably ethanol, were essential for preparation of injection. Ethanol has been regarded as the best biocompatible solvent for taxane derivative like docetaxel. Ethanol is either used as a solvent for docetaxel or as a dilution additive with water for injection to dilute the drug concentrates before administering it to the patient.

Docetaxel injection, marketed as Taxotere, contains two vials in which one vial contains 20 mg docetaxel in 0.5 ml polysorbate 80 and the other vial contains diluent that is 13% w/w ethanol in water for injection.

U.S. Pat. No. 5,438,072 by Rhone-Poulenc claims injectable compositions comprising taxane derivatives in a surface active agent selected from polysorbates, ethylene oxide esters-ethers and fatty acids glycerides, and a water solution of an effective amount of a dilution additive selected from organic compounds having a hydroxyl group an amine functional group and a molecular weight of less than 200 or sodium chloride. So the drug-surfactant concentration is diluted by dilution additive water solution before administering it to the patient.

U.S. Pat. No. 5,750,561 claims injectable solution consisting essentially of docetaxel dissolved in a mixture of ethanol and a polysorbate, which contains up to about 1 mg/ml of docetaxel wherein said injectable solution is capable of being injected without anaphylactic or alcohol intoxication manifestations being associated therewith. The ethanol and polysorbate mixture contains less than 5% each of ethanol and polysorbate.

U.S. Pat. No. 5,698,582 claims compositions comprising a taxane derivative dissolved in a surfactant selected from polysorbate or polyethoxylated castor oil, and essentially free or free of ethanol. The process of preparation of the injectable formulation according to this patent is either by any of the following process:

The first process comprises dissolving taxane derivative in ethanol, and gradually adding the surfactant. The ethanol is then completely or almost completely eliminated. The second process involves dissolving docetaxel in solution comprising surfactant in small amount of ethanol (preferably 1 to 2%).

The patent discloses that presence of small amount of ethanol has several advantages like lower viscosity of surfactant-ethanol solution and improved wetting and filtration of active powder docetaxel.

U.S. Pat. No. 5,714,512 claims a composition, which comprises dissolving docetaxel in a surfactant selected from polysorbate, polyoxyethylated vegetable oil, and polyethoxylated castor oil, said composition being essentially free or free of ethanol. The process of preparing the composition comprises dissolving active ingredient in ethanol, adding surfactant and finally removing the ethanol.

Further patents like US2006188566 claims nanoparticulate docetaxel composition with atleast one surface stabilizer wherein the average particle size of docetaxel is less than 2000 nm.

JP2005225818 claims composition containing paclitaxel or docetaxel with ethanol and polyethylene glycol wherein the ratio of ethanol to polyethylene glycol is 1:4 to 4:1. CN1850056 claims freeze-dried powder injection and process of its preparation. This injection comprises docetaxel, co-solvent (like polyethylene glycol and/or poloxamer, polysorbate 80 or combination of poloxamer and polysorbate 80) and skeleton-supporting agent mannitol.

WO 9528923 claims pharmaceutical composition comprising active principle, which can be docetaxel, at least one unsaturated phospholipid and at least one negative phospholipid, wherein both mentioned phospholipids are different.

Accordingly while ethanol is used as a solvent for docetaxel, it has been eliminated from the final composition to minimize its amount to less than 5%, more preferably within 1-2%. Reducing ethanol from formulation helps to reduce the intoxication drawback of the formulation. Hence, it is accomplished that ethanol is essential for preparation of injection for docetaxel either as a dilution additive with water for injection or solubility enhancer to formulate injection. Although ethanol is essential to enhance solubility of docetaxel, use of ethanol in composition conveys negative aspect like anaphylactic shock or alcohol intoxication.

Further one more drawback is also notified pertaining to stability of composition, which is affected at higher pH. Accordingly as pH goes ahead of 5, compositions turn out to be unstable in consequence of amount of impurities, those are not compatible to ICH guidelines.

Accordingly with an intension to prevent patient from negative aspect of ethanol and attain stable composition of taxane, the inventors of the present invention have made an effort by keeping ethanol absolutely away. In the present invention, ethanol is used neither as a solvent nor as a dilution additive. So the likelihood of anaphylactic shock or alcoholic intoxication is completely avoided upon administration of this injection to a patient.

### OBJECT OF THE INVENTION

The main object of the invention is to originate absolutely ethanol free docetaxel composition to eliminate alcoholic intoxication or anaphylactic shock after administration.

Another object of the invention is to inhibit the development of impurities generated at higher pH by restricting the pH value.

Another object of the invention is to furnish stable docetaxel composition by pH adjustment with support of stabilizers.

One more object of the invention is to provide process for preparation of stable composition of taxane derivative.

### SUMMARY OF THE INVENTION

The present invention provides a stable, absolutely ethanol free, composition of docetaxel to prevent the patient from alcoholic intoxication or anaphylactic shock Further this invention also describes the process for preparation of stable composition of docetaxel.

### DETAILED DESCRIPTION OF THE INVENTION

According to present invention stable composition of taxane derivative comprises mixtures of one or more stabilizers, surfactant, and pharmaceutically acceptable co-solvent.

The present invention is categorized in two embodiments. For that, according to a first embodiment of present invention, is a solution containing an active ingredient was prepared in mixture of surfactant, stabilizer and co-solvent.

While second embodiment includes two vial composition wherein first vial contains product solution of taxane derivative in surfactant and stabilizers and the dilution solution contains co-solvent in water.

The preferable taxane derivative in above embodiments of the present invention is docetaxel wherein the solution comprising docetaxel, dissolved in mixture of one or more stabilizers and pharmaceutically acceptable surfactant, is diluted with one or more diluent additive/co-solvent. The composition has concentration of 5 to 15 mg/ml, which is further diluted with 0.9% NaCl or 5% dextrose solution before administration to the patient. The preferred taxane derivative, docetaxel, is in amount of 10 to 60 mg/ml more preferably 25 to 45 mg/mL.

Stabilizers, for pH adjustment to enhance stability, are either organic acid or inorganic acids wherein the preferred stabilizer is organic acid selected from the group comprising but not limited to citric acid, oxalic acid, lactic acid and the like. The amount of stabilizers used for pH adjustment is quantity sufficient to maintain the pH of the formulation between 2 to 5, more preferably between 3 to 4.

The preferred surfactant used in the injectable formulation of present invention is polysorbate (e.g. Tween), polyoxyethylated vegetable oil (e.g. Emulphor) and polyethoxylated castor oil (e.g. Cremophor). It is used in sufficient quantity to maintain the volume upto 1 ml.

The preferable co-solvent, assisting in inhibition of gel formation, can be propylene glycol, polyethylene glycol (of more than 200 m.wt preferably PEG 400) and the like. The amount of co-solvent is in the range of 10 to 60% w/v, more preferably between 20 to 40% w/v of the formulation.

Further comparative study at accelerated temperature, between proposed invention and composition without stabilizers, as describe below demonstrate use of stabilizer can restrict amount of impurities in finished product and thereby composition attain stability for extended term storage.

| Proposed Invention | | | | Without Stabilizer | | | |
|---|---|---|---|---|---|---|---|
| | | | Total | | | | Total |
| Composition | | Assay | Impurities | Composition | | Assay | Impurities |
| 1. Docetaxel | Initial | 97.0 | 0.186 | 1. Docetaxel | Initial | 98.3 | 0.120 |
| 2. polysorbate 80 | After 3 | 93.6 | 0.620 | 2. Polysorbate 80 | After 15 | 93.1 | 2.873 |
| 3. Citric Acid | months | | | | days | | |

Throughout this specification and the appended claims it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

### Example

The present invention has been described by way of example only, and it is to be recognized that modifications thereto falling within the scope and spirit of the appended claims, and which would be obvious to a person skilled in the art based upon the disclosure herein, are also considered to be included within the scope of this invention.

TABLE 1
The first embodiment is classified with respect to following example.

| Sr. No. | Ingredients | Quantity |
|---|---|---|
| 1. | Docetaxel | 10-60 mg/ml |
| 2. | Co-solvent | 10-60% |
| 3. | Stabilizer | q.s. |
| 4. | Surfactant | q.s. |

### Process of Preparation:

This stable, composition can be prepared by either of the following processes:

Process-I: It comprises the following steps:
a) Make a mixture of surfactant and stabilizers till desired pH.
b) Add co-solvent to the solution of step-a).
c) Add docetaxel to the solution of step b).
d) Filter the solution of step-iv through 0.2-micron filter.
e) After the filtration, fill the solution into vial.

Process-II: It comprises the following steps:
a) Make a mixture of surfactant and co-solvent.
b) Add stabilizer, to adjust pH, to the solution of step-a),
c) Add docetaxel to the solution of step b)
d) Filter the solution of step-c through 0.2-micron filter.
e) After the filtration, fill the solution into vial.

The solution prepared as either of the process is taken from the vial and diluted with water for injection for further use.

### Example I

| Sr. No. | Ingredients | Quantity |
|---|---|---|
| 1. | Docetaxel | 40 mg/ml |
| 2. | Polysorbate 80 | q.s. |
| 3. | Citric acid | q.s. |
| 4. | PEG 400 | 300 mg/mL |

### Process of Preparation:

The composition classified in example I can be prepared by either of the process disclose above

TABLE II
The second embodiment of the invention is classified
with respect to the following examples.

| Sr. No. | Ingredients | Quantity |
|---|---|---|
| 1. | Docetaxel | 10-60 mg/ml |
| 2. | Stabilizer | q.s |
| 3. | Surfactant | q.s |

This injectable formulation can be prepared by following process:
a) Make mixture of surfactant and stabilizer
b) Add docetaxel to solution of step a) to get product solution.
c) Filter the solution of step-b) through 0.2-micron filter.
d) After the filtration, fill the solution into vial.

The product solution prepared as per the above process is taken from the vial and is diluted by water for injection or mixture of water for injection and co-solvent.

### Example II

| Sr. No. | Ingredients | % Range |
|---|---|---|
| 1. | Docetaxel | 40 mg/ml |
| 2. | Citric acid | q.s |
| 3. | Polysorbate 80 | q.s to 1 mL |

The product solution prepared as per the above process is taken from the vial and is diluted by water for injection or mixture of water for injection and polyethylene glycol having molecular weight higher than 200, preferably PEG 400. The above said embodiments of the invention can be illustrated but not limited to above example(s).

1. Stable injectable composition comprising taxane derivative wherein said composition comprising mixture of docetaxel, one or more stabilizer, one or more surfactant, or more co-solvent and water for injection wherein said composition is absolutely free of ethanol.

2. Stable injectable composition as claimed in claim 1, wherein surfactant is selected from the group consisting of polysorbate, polyoxyethylated vegetable oil and polyethoxylated castor oil.

3. Stable injectable composition as claimed in claim 1, wherein co-solvent is selected from the group comprising propylene glycol and polyethylene glycol having molecular weight more than 200.

4. Stable injectable composition as claimed in claim 1, wherein stabilizer is organic acid selected from the group comprising citric acid, oxalic acid and lactic acid.

5. Stable injectable composition as claimed in claim 1, wherein the said composition comprises docetaxel, dissolved in mixture of one or more stabilizers, pharmaceutically acceptable surfactant and one or more co-solvent.

6. Stable injectable composition of taxane derivative as claimed in claim 1, wherein the said composition comprises of 1 0-60-mg/mL docetaxel, one or more stabilizers to attain the desired pH, surfactant and 10-60% w/v co-solvent.

7. Stable injectable composition of taxane derivative as claimed in claim 1, wherein product solution comprises 10-60 mg/mL docetaxel, stabilizer to attain the desired pH, surfactant and dilution solution comprising mixture of co-solvent and water for injection wherein co-solvent is of 10-60% w/v.

8. Stable injectable composition of taxane derivative comprising product solution and dilution solution, wherein the product solution comprises docetaxel dissolved in polysorbate 80 followed by addition of sufficient amount of citric acid to attain the desired pH and dilution solution comprises polyethylene glycol with molecular weight more than 200 in water.

9. Stable injectable composition of taxane derivative as claimed in claim 1, wherein the desired pH is between 2-5.

10. (canceled)

11. Stable injectable composition of taxane derivative as claimed in claim 8, wherein the desired pH is between 2-5.
